(19) United States
(12) Patent Application Publication
(10) Pub. No.: US 2026/0128150 A1
(43) Pub. Date: May 7, 2026

(54) HEALTHCARE RECOMMENDATION METHOD AND SYSTEM USING REHABILITATION DEVICE

(71) Applicant: INSTITUTE FOR INFORMATION INDUSTRY, Taipei City (TW)

(72) Inventors: Chih Lin WU, Taipei City (TW); Yun Cheng JHONG, Taipei City (TW); Zhi Ying CHEN, Taipei City (TW); Chien Der LIN, Taipei City (TW)

(73) Assignee: INSTITUTE FOR INFORMATION INDUSTRY, Taipei City (TW)

(21) Appl. No.: 18/958,247

(22) Filed: Nov. 25, 2024

(30) Foreign Application Priority Data
Nov. 6, 2024 (TW) 113142537

(51) Int. Cl. G16H 20/30

(52) U.S. Cl. CPC G16H 20/30

(57) ABSTRACT

The present disclosure provides a healthcare recommendation method and system using rehabilitation device, wherein the healthcare recommendation method, performed by a processing device, includes: obtaining raw sensing data from sensing devices, labeling one of the raw sensing data according to personalized feature labels to generate labeled data, inputting one of the raw sensing data to a pre-trained predication model to generate predicted data, performing data fusion on the labeled data and the predicted data to generate fusion data, extracting multi-dimensional feature data from the raw sensing data, inputting the fusion data and the multi-dimensional feature data into a pre-trained inference model to generate an exercise effectiveness indicator, and generating a healthcare plan based on the exercise effectiveness indicator.

### CROSS-REFERENCE TO RELATED APPLICATIONS

This non-provisional application claims priority under 35 U.S.C. § 119(a) on Patent Application No(s). 113142537 filed in Republic of China (Taiwan) on Nov. 6, 2024, the entire contents of which are hereby incorporated by reference.

### BACKGROUND

### 1. Technical Field

This disclosure relates to a healthcare recommendation method and system.

### 2. Related Art

Population aging has become a global trend and is progressing rapidly. Therefore, how to effectively care for the aging population in the future will be a significant challenge for society. In response to the aging society, caregiving institutions need to integrate and utilize information and communication technology products and services to address the issue of manpower shortages, while also improving the quality of care for the elderly.

In the current rehabilitation care process, the assessment of operating rehabilitation device is primarily based on the number of operations and duration or the use of general standard models for quantitative evaluation. However, these methods often overlook an individual's physical condition, the operation process, and personal health historical data, making it impossible to conduct personalized effectiveness evaluation based on multi-dimensional data, which limits the accuracy and specificity of rehabilitation outcomes.

### SUMMARY

Accordingly, this disclosure provides a healthcare recommendation method and system.

According to one or more embodiment of this disclosure, a healthcare recommendation method, performed by a processing device, includes: obtaining a plurality of raw sensing data from a plurality of sensing devices; labeling one of the raw sensing data according to a plurality of personalized feature labels to generate labeled data; inputting one of the raw sensing data into a pre-trained prediction model to generate predicted data; performing data fusion on the labeled data and the predicted data to generated fused data; extracting multi-dimensional feature data from the raw sensing data; inputting the fused data and the multi-dimensional feature data into a pre-trained inference model to generate an exercise effectiveness indicator; and generating a healthcare plan based on the exercise effectiveness indicator.

According to one or more embodiment of this disclosure, a healthcare recommendation system includes: a plurality of sensing devices and a processing device. The sensing devices are configured to obtain a plurality of raw sensing data. The processing device is connected to the sensing devices. The processing device is configured to label one of the raw sensing data according to a plurality of personalized feature labels to generate labeled data, input one of the raw sensing data into a pre-trained prediction model to generate predicted data, perform data fusion on the labeled data and the predicted data to generated fused data, extract multi-dimensional feature data from the raw sensing data, input the fused data and the multi-dimensional feature data into a pre-trained inference model to generate an exercise effectiveness indicator, and generate a healthcare plan based on the exercise effectiveness indicator.

In view of the above, the healthcare recommendation method and system according to an embodiment of the present disclosure may perform personalized labeling on sensing data, feature extraction on multi-dimensional data and processing multiple model data, perform assessment of personalized exercise effectiveness indicator by using the above data, and provide the healthcare plan to the user accordingly. Therefore, the issue of staff shortages in care institutions may be resolved, and the quality of care may be improved.

### BRIEF DESCRIPTION OF THE DRAWINGS

The present disclosure will become more fully understood from the detailed description given herein below and the accompanying drawings which are given by way of illustration only and thus are not limitative of the present disclosure and wherein:

FIG. 1 is a block diagram illustrating a healthcare recommendation system according to an embodiment of the present disclosure;

FIG. 2 is a flow chart illustrating a healthcare recommendation method according to an embodiment of the present disclosure;

FIG. 3 is a flow chart illustrating a movement smoothness analysis in the healthcare recommendation method according to an embodiment of the present disclosure;

FIG. 4 is a flow chart illustrating building personalized features in the healthcare recommendation method according to an embodiment of the present disclosure;

FIG. 5 is a flow chart illustrating obtaining a pre-trained inference model in the healthcare recommendation method according to an embodiment of the present disclosure; and

FIG. 6 is a flow chart illustrating obtaining the pre-trained inference model in the healthcare recommendation method according to another embodiment of the present disclosure.

### DETAILED DESCRIPTION

In the following detailed description, for purposes of explanation, numerous specific details are set forth in order to provide a thorough understanding of the disclosed embodiments. According to the description, claims and the drawings disclosed in the specification, one skilled in the art may easily understand the concepts and features of the present invention. The following embodiments further illustrate various aspects of the present invention, but are not meant to limit the scope of the present invention.

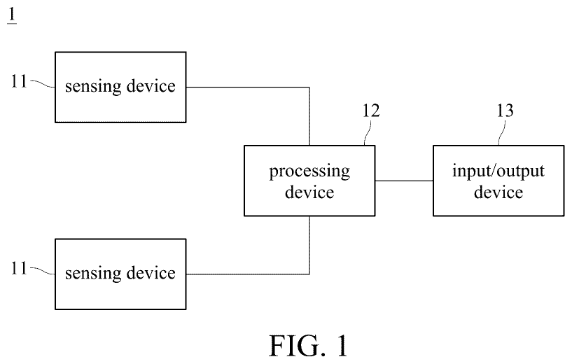

Please refer to FIG. 1, wherein FIG. 1 is a block diagram illustrating a healthcare recommendation system according to an embodiment of the present disclosure. As shown in FIG. 1, the healthcare recommendation system 1 includes sensing devices 11, a processing device 12 and an input/output device 13. The processing device 12 is connected to the sensing devices 11 and the input/output device 13 in a wired or wireless manner. The input/output device 13 is configured to provide a healthcare plan to a user. It should be noted that FIG. 1 exemplarily shows two sensing devices 11, but the number of the sensing devices may be more than two. The present disclosure does not limit the number of the sensing devices.

Each of the sensing devices 11 may be a motion sensing device, a physiological sensing device, other sensing device for movement performance or physical condition of the user and a combination of the sensing devices described above. The sensing devices 11 are configured to sense the movement and/or physical condition of the user to generate raw sensing data. The motion sensing device may include one or more of a distance sensor, a velocity sensor and an angle sensor, the corresponding raw sensing data may include one or more of exercise time, a motion trajectory and somatosensory interactive data. Further, the motion trajectory may be obtained according to a moving distance, a moving velocity and a rotation angle. The motion sensing device may be installed on the user or rehabilitation device (for example, rehabilitation exercise equipment) operated by the user to obtain the movement trajectory and the corresponding time point(s) of the user. The physiological sensing device may include one or more of a blood pressure sensor, a blood oxygen sensor, a blood sugar sensor, a body temperature sensor and a body fat and weight sensor. The physiological sensing device includes but not limited to the physiological information sensors listed above. The corresponding raw sensing data may include blood pressure, blood oxygen concentration, blood sugar concentration, body temperature, body fat and weight. The raw sensing data includes but not limited to the items listed above. The physiological sensing device may be installed on the user.

The processing device 12 is configured to determine the exercise effectiveness of the user according to the sensing results of the sensing devices 11, thereby recommending the corresponding healthcare plan to the user. The processing device 12 may include one or more processors, the processor is, for example, a central processing unit, a graphics processing unit, a microcontroller, a programmable logic controller or any other processor with signal processing functions.

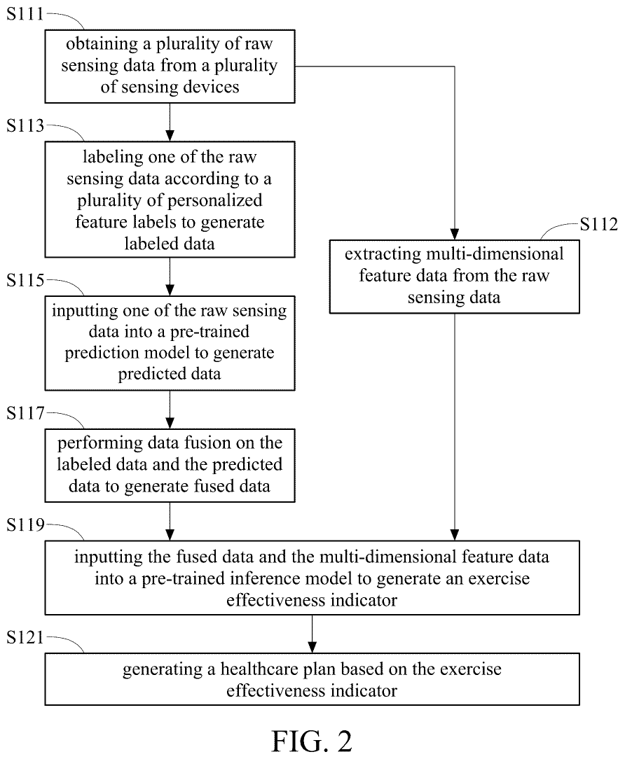

Please refer to FIG. 1 and FIG. 2, wherein FIG. 2 is a flow chart illustrating a healthcare recommendation method according to an embodiment of the present disclosure. As shown in FIG. 2, the healthcare recommendation method includes step S111-step S121.

In step S111, the processing device 12 obtains a plurality of raw sensing data from the sensing devices 11 sensing the motion and/or physical condition of the user. The raw sensing data may indicate the exercise condition and/or physical condition of the user during exercise.

In step S112, the processing device 12 extracts multi-dimensional feature data from the raw sensing data. The multi-dimensional feature data may indicate data of the raw sensing data in the same time period. In the embodiment of one sensing device generating one type of raw sensing data, the number of the dimensions may correspond to the number of the sensing device. The processing device 12 may extract the part corresponding to the same time period of different types of raw sensing data generated by different types of sensing devices 11, and utilize the extracted part as the multi-dimensional feature data. For example, the raw sensing data may include, but not limited to, motion sensing data, physiological sensing data, physical fitness data, movement recognition data, voice recognition data, etc. In step S112, the processing device 12 simultaneously considers the features of multiple raw sensing data to create personalized multi-dimensional feature data.

In step S113, the processing device 12 utilizes personalized feature labels to label one of the raw sensing data to generate labeled data. In an embodiment, the processing device 12 may utilize threshold-based auto-labeling (TBAL) algorithm to perform labeling according to a given threshold and waveform features corresponding to various movement features. The threshold may indicate the raw sensing data is determined as valid data, and the waveform feature may be the waveform feature of the raw sensing data. The personalized feature labels may include areas of exercise and corresponding movement features, such as upper limb abduction, upper limb pulling back, lower limb squatting, and lower limb knee-raising, etc. The present disclosure does not limit the exercise area and movement feature.

In step S115, the processing device 12 inputs one of the raw sensing data into a pre-trained prediction model to generate predicted data. The pre-trained prediction model is a model that is pre-trained with motion-related parameters, and the pre-trained prediction model may include, but not limited to, one or more of a calorie prediction model, an exercise intensity prediction model, and a muscular endurance prediction model, etc. The predicted data is the result predicted by the pre-trained prediction model, and the predicted data may include, but not limited to, calories burned, applied strength, and level of muscular endurance, etc. It should be noted that the raw sensing data labeled in step S113 and the raw sensing data input to the pre-trained prediction model in step S115 may be the same piece of data or different pieces of data.

In step S117, the processing device 12 performs data fusion on the labeled data and the predicted data to generate fused data, wherein the fused data may be metadata. Data fusion may be implemented by observation-level fusion, feature-level fusion, and/or decision-level fusion, which is not limited in the present disclosure.

In step S119, the processing device 12 inputs the fused data and the multi-dimensional feature data into a pre-trained inference model to generate an exercise effectiveness indicator. The exercise effectiveness indicator may be presented in the form of score, percentage, etc. The exercise effectiveness indicator may be the analysis result of the exercise condition and physical condition of the user.

In step S121, the processing device 12 generates and outputs the healthcare plan according to the exercise effectiveness indicator. For example, when the healthcare recommendation method and system according to an embodiment of the present disclosure is applied in rehabilitation, the healthcare plan may be generated through health promotion effectiveness analysis. The health promotion effectiveness analysis may be combined with various data (for example, pre-and post-test physical fitness assessment data, somatosensory interactive data, and movement smoothness analysis data, etc.) obtained in the previous steps to analyze user's physical condition, thereby providing the user with recovery indicator and recommendation for health promotion. The healthcare plan may include dynamic prescription and/or personalized healthcare plan, wherein the dynamic prescription may be a rehabilitation prescription dynamically adjusted by the processing device 12 according to the result of health promotion analysis and provided to the user. The personalized healthcare plan may include the combination of the result of health promotion effectiveness analysis and the dynamic prescription, thereby providing the user with a complete training plain and recommendation.

The healthcare recommendation method and system according to an embodiment of the present disclosure may perform personalized labeling on sensing data, feature extraction on multi-dimensional data and processing multiple model data, perform assessment of personalized exercise effectiveness indicator by using the above data, and provide the healthcare plan to the user accordingly. Therefore, the issue of staff shortages in care institutions may be resolved, and the quality of care may be improved.

In an embodiment, the processing device 12 may be further configured to obtain user feedback data through the input/output device 13, and adjust the threshold setting used in the labeling described in step S113 according to the user feedback data. The user feedback data may include, but not limited to, rate of perceived exertion (PRE) filled out by the user, physiological signal of the user, test result of fitness assessment, etc. The threshold setting may indicate the movement goal to be achieved, including but not limited to amplitude threshold, frequency threshold, slope threshold, time interval threshold, etc. For example, when the user feedback data shows that the current exercise intensity exceeds the user's tolerance, the processing device 12 may lower the threshold setting. Conversely, when the user feedback data shows that the current exercise intensity is below the user's tolerance, the processing device 12 may increase the threshold setting. Therefore, even if the user's tolerated exercise intensity is lower, the threshold may be lowered to allow the user to complete exercises that match their physical condition. Conversely, when the user's tolerated exercise intensity is higher, the threshold may be increased to enable the user to challenge exercises suited to their physical condition.

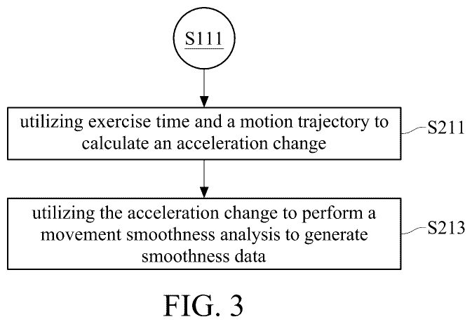

Please refer to FIG. 1 and FIG. 3, wherein FIG. 3 is a flow chart illustrating a movement smoothness analysis in the healthcare recommendation method according to an embodiment of the present disclosure. In the embodiment in FIG. 3, the raw sensing data may include the exercise time and the motion trajectory. As shown in FIG. 3, the movement smoothness analysis includes step S211 and S213.

In step S211, the processing device 12 may utilize the exercise time and the motion trajectory to calculate an acceleration change. The processing device 12 may calculate the change in acceleration over time of the motion trajectory relative to the exercise time (the derivative of acceleration).

In step S213, the processing device 12 may utilize the acceleration change to perform a movement smoothness analysis to generate smoothness data. The smoothness data may be presented in the form of score. For example, when the acceleration change is smaller, the score of the smoothness data is higher, and vice versa. Specifically, when the movement is fast, consistent, and smooth, it indicates that the user has good physical ability. Generally, younger individuals tend to have higher smoothness compared to older individuals, resulting in lower acceleration change. Conversely, older individuals have lower movement smoothness, leading to higher acceleration change.

Accordingly, the sensing device may collect the user's movement time of the rehabilitation device and the movement amount of the user's motion trajectory during rehabilitation activities. Through the calculation mechanism of varying acceleration, the smoothness of the user's operation when using the rehabilitation device may be obtained.

The processing device 12 may utilize the smoothness data as one of the pieces of data for the pre-trained inference model to generate the exercise effectiveness indicator. Step S211 and step S213 may be performed, but not limited to, between step S111 and step S117 in FIG. 2. Specifically, step S117 in FIG. 2 may include perform data fusion on the labeled data, the predicted data and the smoothness data to generate the fused data.

Further, step S213 in FIG. 3 may include obtaining a motion type corresponding to the motion trajectory, and selecting one of movement smoothness analysis algorithms according to the motion type to perform the movement smoothness analysis. Specifically, the processing device 12 may determine whether the motion type is a linear motion or a rotational motion based on the motion trajectory, and select the corresponding movement smoothness analysis algorithm according to the motion type to perform the movement smoothness analysis. The movement smoothness analysis algorithms may include the log dimensionless jerk (LDLJ) algorithm and the Spitzer photometry and accurate rotation curve (SPARC) algorithm. When the motion type is the linear motion, the processing device 12 may use any one of the LDLJ algorithm and the SPARC algorithm, wherein the SPARC algorithm is preferable; and when the motion type is the rotational motion, the processing device 12 may use the LDLJ algorithm.

In an embodiment, the processing device 12 using the SPARC algorithm may obtain the smoothness by calculating the curve length of the Fourier spectrum of a given velocity curve within the frequency range of 0 Hz to 20 Hz. Additionally, the processing device 12 may adjust the cut-off frequency by changing the threshold. For waveform with more noise, a higher threshold may be adopted to improve measurement accuracy, while a lower threshold may be adopted to provide more motion detail. Through preliminary testing, an automatic or semi-automatic adjustment of the threshold may achieve a balance between accuracy and stability, allowing for the acquisition of appropriate smoothness data.

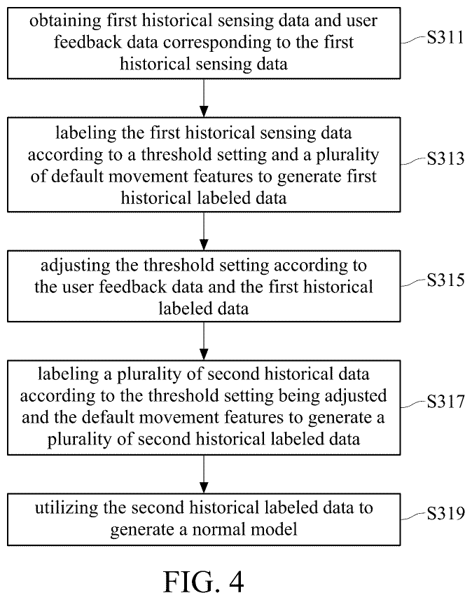

Please refer to FIG. 1 and FIG. 4, wherein FIG. 4 is a flow chart illustrating building personalized features in the healthcare recommendation method according to an embodiment of the present disclosure. As shown in FIG. 4, building a normal model includes step S311-step S319. Steps shown in FIG. 4 may be performed before labeling the raw sensing data according to the personalized feature labels (step S115 in FIG. 2), but the present disclosure is not limited thereto. Steps in FIG. 4 may be performed by the processing device 12 in FIG. 1, or by another processing device, and said another processing device may obtain the normal model and then output the normal model to the processing device 12.

In step S311, first historical sensing data obtained by the processing device 12 may be data coming from the sensing devices 11, the generation timing of the first historical sensing data may be earlier than the generation timing of the raw sensing data. The first historical sensing data may be unlabeled and unprocessed data. The user feedback data of the first historical sensing data may be the same data type as the user feedback data described above. The user feedback data of the first historical sensing data is a feedback of the user on the exercise corresponding to the first historical sensing data.

In step S313, the processing device 12 labels the first historical sensing data according to the threshold setting and default movement features to generate first historical labeled data. The default movement features may include areas of exercise and corresponding movement features, which are the same as the areas of exercise and the corresponding movement features described above. The threshold setting corresponding to the default movement features may be an initial setting, and the threshold setting corresponding to the personalized feature labels described above may be the adjusted initial setting. The first historical labeled data may include the first historical sensing data and the corresponding feature labels. The processing device 12 may use the TBAL algorithm to label the first historical sensing data.

In step S315, the processing device 12 may adjust the threshold setting according to the user feedback data and the first historical labeled data. In other words, the processing device 12 may adjust the threshold setting for using the default movement features for labeling according to the user feedback data. The implementation of step S315 may be the same as adjusting the threshold setting according to the personalized feature labels described above, and repeated descriptions are omitted herein.

In step S317, the processing device 12 may label second historical sensing data according to the adjusted threshold setting and the default movement features to generate second historical labeled data. The second historical sensing data may be the same data type as the first historical sensing data described above. Each of the default movement features may corresponding to the second historical sensing data, and the second historical sensing data of each of the default movement features may have different levels of completeness. The second historical sensing data and the first historical sensing data may have the same or different generation timing. The difference between the first historical sensing data and the second historical sensing data is that the first historical sensing data is used for adjusting the threshold setting, while the second historical sensing data is used for building the normal model described below. The implementation of step S317 may be the same as step S313, and repeated descriptions are omitted herein.

In step S319, the processing device 12 may utilize the second historical labeled data to generate the normal model, wherein the normal model indicate the personalized features. The normal model may be a classification model, such as a model based on logistic regression, decision tree, random forest, etc., but the present disclosure is not limited thereto. Specifically, the normal model may include the default movement features along with waveform features corresponding to various levels of completeness for each default movement feature. For example, the normal model may include waveform features of a specific movement at completeness levels ranging from 0% to 100%. In an implementation, the processing device 12 may directly utilize the second historical labeled data and the classification algorithm to build the normal model. In another implementation, the processing device 12 may first utilize the second historical labeled data and one or more classification algorithms to generate one or more initial models, and verify the initial models (for example, through cross-validation, bootstrapping, etc.). If the validation result indicates that the performance of the initial model does not meet the expected performance, the threshold setting is adjusted again, and the process is restarted from step S313. This continues until the validation result indicates that the performance of the initial model meets the expected performance, and the processing device 12 may adopt the initial model as the normal model. After generating the normal model, the processing device 12 may utilize the normal model to correct the labeled data to automatically adjust the threshold.

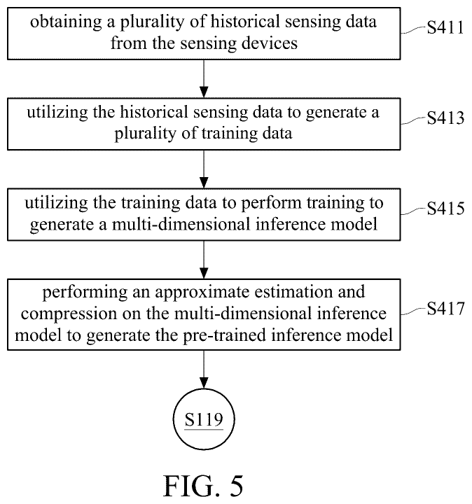

Please refer to FIG. 1 and FIG. 5, wherein FIG. 5 is a flow chart illustrating obtaining the pre-trained inference model in the healthcare recommendation method according to an embodiment of the present disclosure. As shown in FIG. 5, obtaining the pre-trained inference model includes step S411-step S417. Steps shown in FIG. 5 may be performed before inputting the fused data and the multi-dimensional feature data into the pre-trained inference model to generate the exercise effectiveness indicator (step S119 in FIG. 2), but the present disclosure is not limited thereto. It should be noted that steps in FIG. 5 may be performed by the processing device 12 in FIG. 1, or by another processing device, and said another processing device may obtain the pre-trained inference model and then output the pre-trained inference model to the processing device 12. For the convenience of description, the following uses the processing device 12 to describe the steps in FIG. 5.

The implementation of performing step S411 to obtain historical sensing data may be the same as obtaining the raw sensing data in step S111 in FIG. 2, except that the generation timing of the historical sensing data may precede that of the raw sensing data.

In step S413, the processing device 12 may perform data fusion on the historical sensing data and extract multi-dimensional feature data corresponding to time periods, and assign the corresponding exercise effectiveness indicator to each of the multi-dimensional feature data to utilize the assigned multi-dimensional feature data as training data.

In step S415, the processing device 12 may utilize the training data to perform training to generate a multi-dimensional inference model.

In step S417, the processing device 12 may perform an approximate estimation and compression on the multi-dimensional inference model to generate the pre-trained inference model. The processing device 12 may convert the logic of the multi-dimensional inference model into a numerical analysis workflow, and optimize parameters using numerical analysis methods (such as Lait, least squares, Lagrange, etc.). Based on the optimization results, the processing device 12 then assesses the impact of constraints in optimization, and finally performs predictive analysis. Furthermore, the processing device 12 may calculate the weight of a layer in the multi-dimensional inference model as smaller bits to represent each weight, then store the corresponding weights using a clustering algorithm, wherein more frequently occurring symbol may be represented by shorter bits. Through approximate estimation and compression, important connections may be retained, resulting in a sparse network structure.

Accordingly, the pre-trained inference model generated through the above approach may be implemented on edge devices, enabling efficient and low-power computation. By the inference model on edge devices, personalized healthcare plan may be generated. During the inference process of the pre-trained inference model, the pre-trained inference model may group weights with similar values and remove features that contribute less to the inference result, further reducing computational and storage requirements.

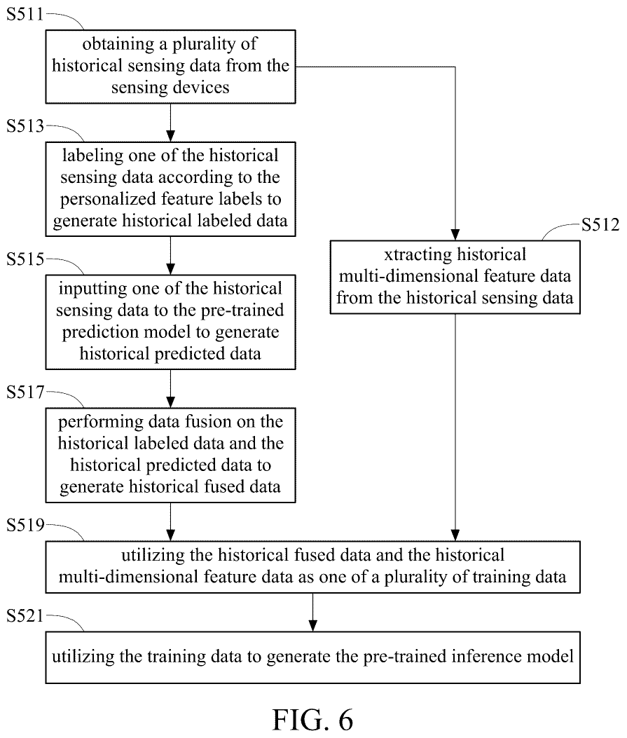

Please refer to FIG. 1 and FIG. 6, wherein FIG. 6 is a flow chart illustrating obtaining the pre-trained inference model in the healthcare recommendation method according to another embodiment of the present disclosure. As shown in FIG. 6, obtaining the pre-trained inference model includes step S511-step S521. Steps shown in FIG. 6 may be performed before inputting the fused data and the multi-dimensional feature data into the pre-trained inference model to generate the exercise effectiveness indicator (step S119 in FIG. 2), but the present disclosure is not limited thereto. Step S511 may be the same as step S411 in FIG. 5; the implementations of steps S512, S513, S515, and S517 may be the same as steps S112, S113, S115, and S117 in FIG. 2, respectively, and repeated descriptions are omitted herein. It should be noted that steps in FIG. 6 may be performed by the processing device 12 in FIG. 1, or by another processing device, and said another processing device may obtain the pre-trained inference model and then output the pre-trained inference model to the processing device 12. For the convenience of description, the following uses the processing device 12 to describe the steps in FIG. 6.

In step S519, the processing device 12 may utilize historical fused data and historical multi-dimensional feature data as one piece of training data. In step S521, the processing device 12 may utilize the training data to perform training to generate the multi-dimensional inference model. The multi-dimensional inference model may be used to generate the healthcare plan described above. In an implementation, step S521 in FIG. 6 may be implemented by step S415 and step S417 in FIG. 5.

Through the healthcare recommendation method and system according to one or more embodiments describe above, the issues currently faced in quantifying rehabilitation exercise outcomes may be addressed. Using sensing data from rehabilitation device, a personalized normal model may be generated to enable multifaceted, multi-dimensional feature extraction, thereby creating a personalized healthcare plan. Additionally, the burden on healthcare providers may also be reduced.

In view of the above, the healthcare recommendation method and system according to an embodiment of the present disclosure may perform personalized labeling on sensing data, feature extraction on multi-dimensional data and processing multiple model data, perform assessment of personalized exercise effectiveness indicator by using the above data, and provide the healthcare plan to the user accordingly. Therefore, the issue of staff shortages in care institutions may be resolved, and the quality of care may be improved. Further, by adjusting the threshold setting according to the user feedback data, even if the user's tolerated exercise intensity is lower, the threshold may be lowered to allow the user to complete exercises that match their physical condition. Conversely, when the user's tolerated exercise intensity is higher, the threshold may be increased to enable the user to challenge exercises suited to their physical condition. The pre-trained inference model generated through approximate estimation and compression may be implemented on edge devices, enabling efficient and low-power computation. By the inference model on edge devices, personalized healthcare plan may be generated. During the inference process of the pre-trained inference model, the pre-trained inference model may group weights with similar values and remove features that contribute less to the inference result, further reducing computational and storage requirements.

What is claimed is:

1. A healthcare recommendation method, performed by a processing device, comprising:
obtaining a plurality of raw sensing data from a plurality of sensing devices;
labeling one of the plurality of raw sensing data according to a plurality of personalized feature labels to generate labeled data;
inputting one of the plurality of raw sensing data into a pre-trained prediction model to generate predicted data;
performing data fusion on the labeled data and the predicted data to generated fused data;
extracting multi-dimensional feature data from the plurality of raw sensing data;
inputting the fused data and the multi-dimensional feature data into a pre-trained inference model to generate an exercise effectiveness indicator; and
generating a healthcare plan based on the exercise effectiveness indicator.

2. The healthcare recommendation method according to claim 1, wherein the plurality of raw sensing data comprises exercise time and a motion trajectory, and the healthcare recommendation method further comprises:
utilizing the exercise time and the motion trajectory to calculate an acceleration change; and
utilizing the acceleration change to perform a movement smoothness analysis to generate smoothness data.

3. The healthcare recommendation method according to claim 2, wherein performing the data fusion on the labeled data and the predicted data to generated the fused data further comprises:
performing the data fusion on the labeled data, the predicted data and the smoothness data to generate the fused data.

4. The healthcare recommendation method according to claim 2, wherein using the acceleration change to perform the movement smoothness analysis to generate the smoothness data comprises:
obtaining a motion type corresponding to the motion trajectory; and
selecting one of a plurality of movement smoothness analysis algorithms according to the motion type to perform the movement smoothness analysis.

5. The healthcare recommendation method according to claim 1, further comprising:
obtaining user feedback data; and
adjusting a threshold setting of the labeling according to the plurality of personalized feature labels according to the user feedback data.

6. The healthcare recommendation method according to claim 1, further comprising:
obtaining first historical sensing data and user feedback data corresponding to the first historical sensing data;
labeling the first historical sensing data according to a threshold setting and a plurality of default movement features to generate first historical labeled data;
adjusting the threshold setting according to the user feedback data and the first historical labeled data;
labeling a plurality of second historical data according to the threshold setting being adjusted and the plurality of default movement features to generate a plurality of pieces of second historical labeled data; and
utilizing the plurality of second historical labeled data to generate a normal model, wherein the normal model indicates the plurality of personalized feature labels.

7. The healthcare recommendation method according to claim 1, further comprising:
obtaining a plurality of historical sensing data from the plurality of sensing devices;
utilizing the plurality of historical sensing data to generate a plurality of pieces of training data;
utilizing the plurality of training data to perform training to generate a multi-dimensional inference model; and
performing an approximate estimation and compression on the multi-dimensional inference model to generate the pre-trained inference model.

8. The healthcare recommendation method according to claim 1, further comprising:
obtaining a plurality of historical sensing data from the plurality of sensing devices;
labeling one of the plurality of historical sensing data according to the plurality of personalized feature labels to generate historical labeled data;
inputting one of the plurality of historical sensing data to the pre-trained prediction model to generate historical predicted data;
performing data fusion on the historical labeled data and the historical predicted data to generate historical fused data;
extracting historical multi-dimensional feature data from the plurality of pieces of historical sensing data;
utilizing the historical fused data and the historical multi-dimensional feature data as one of a plurality of training data; and
utilizing the plurality of training data to generate the pre-trained inference model.

9. A healthcare recommendation system, comprising:
a plurality of sensing devices configured to obtain a plurality of raw sensing data; and
a processing device connected to the plurality of sensing devices, and configured to label one of the plurality of raw sensing data according to a plurality of personalized feature labels to generate labeled data, input one of the plurality of raw sensing data into a pre-trained prediction model to generate predicted data, perform data fusion on the labeled data and the predicted data to generated fused data, extract multi-dimensional feature data from the plurality of raw sensing data, input the fused data and the multi-dimensional feature data into a pre-trained inference model to generate an exercise effectiveness indicator, and generate a healthcare plan based on the exercise effectiveness indicator.

10. The healthcare recommendation system according to claim 9, wherein the plurality of raw sensing data comprises exercise time and a motion trajectory, and the processing device is further configured to utilize the exercise time and the motion trajectory to calculate an acceleration change, and utilize the acceleration change to perform a movement smoothness analysis to generate smoothness data.

11. The healthcare recommendation system according to claim 10, wherein the processing device is further configured to perform the data fusion on the labeled data, the predicted data and the smoothness data to generate the fused data.

12. The healthcare recommendation system according to claim 10, wherein the processing device is further configured to obtain a motion type corresponding to the motion trajectory, and select one of a plurality of movement smoothness analysis algorithms according to the motion type to perform the movement smoothness analysis.

13. The healthcare recommendation system according to claim 9, wherein the processing device is further configured to obtain user feedback data, and adjust a threshold setting of the labeling according to the plurality of personalized feature labels according to the user feedback data.

14. The healthcare recommendation system according to claim 9, wherein the processing device is further configured to:
obtain first historical sensing data and user feedback data corresponding to the first historical sensing data;
label the first historical sensing data according to a threshold setting and a plurality of default movement features to generate first historical labeled data;
adjust the threshold setting according to the user feedback data and the first historical labeled data;
label a plurality of second historical data according to the threshold setting being adjusted and the plurality of default movement features to generate a plurality of second historical labeled data; and
utilize the plurality of second historical labeled data to generate a normal model, wherein the normal model indicates the plurality of personalized feature labels.

15. The healthcare recommendation system according to claim 9, wherein the processing device is further configured to:
obtain a plurality of historical sensing data from the plurality of sensing devices;
utilize the plurality of historical sensing data to generate a plurality of training data;
utilize the plurality of training data to perform training to generate a multi-dimensional inference model; and
perform an approximate estimation and compression on the multi-dimensional inference model to generate the pre-trained inference model.

16. The healthcare recommendation system according to claim 9, wherein the processing device is further configured to:
obtain a plurality of historical sensing data from the plurality of sensing devices;
label one of the plurality of historical sensing data according to the plurality of personalized feature labels to generate historical labeled data;
input one of the plurality of historical sensing data to the pre-trained prediction model to generate historical predicted data;
perform data fusion on the historical labeled data and the historical predicted data to generate historical fused data;
extract historical multi-dimensional feature data from the plurality of historical sensing data;
utilize the historical fused data and the historical multi-dimensional feature data as one of a plurality of training data; and
utilize the plurality of training data to generate the pre-trained inference model.
